(19) United States
(12) Patent Application Publication
(10) Pub. No.: US 2019/0002367 A1
(43) Pub. Date: Jan. 3, 2019

(54) Systems and Methods for Producing Naphthalenes and Methylnaphthalenes

(71) Applicant: ExxonMobil Chemical Patents Inc., Baytown, TX (US)

(72) Inventors: Michael Salciccioli, Houston, TX (US); Alan A. Galuska, Huffman, TX (US); Tan-Jen Chen, Seattle, WA (US); Catherine M. Dorsi, Houston, TX (US); Mayank Shekhar, Houston, TX (US)

(21) Appl. No.: 15/974,158

(22) Filed: May 8, 2018

Related U.S. Application Data
(60) Provisional application No. 62/525,793, filed on Jun. 28, 2017.

(51) Int. Cl. C07C 2/42; C07C 6/06; C07C 4/18

(52) U.S. Cl. CPC C07C 2/42; C07C 6/06; C07C 2521/04; C07C 2529/40; C07C 2529/08; C07C 4/18

(57) ABSTRACT

A process for producing naphthalene or methylnaphthalenes from an alkane-containing stream. In an embodiment, the produce includes providing an alkane-containing feed stream to a reactor, and contacting the ethane-containing stream with an aromatization catalyst within the reactor. The aromatization catalyst comprises molecular sieve, and a dehydrogenation component. In addition, the process includes producing a reactor effluent stream from the reactor, and separating a product stream from the reactor effluent stream. The product stream comprises at least one or both of naphthalene and methylnaphthalene.

### CROSS-REFERENCE OF RELATED APPLICATIONS

This application claims the benefit of Provisional Application No. 62/525,793, filed Jun. 28, 2017, and entitled “Systems and Methods for Producing Naphthalenes and Methylnaphthalenes,” the disclosure of which is hereby incorporated by reference in its entirety.

### TECHNICAL FIELD

This application generally relates to the production of naphthalenes and methylnaphthalenes. More particularly, this application relates to the production of naphthalenes and methylnaphthalenes from alkane-containing feed streams.

### BACKGROUND

Naphthalenes and methylnaphthalenes are valuable and useful chemical compounds. For example, both naphthalenes and methylnaphthalenes are used in the production of plasticizers (e.g., for concrete) and speciality chemical products. In addition, naphthalenes are utilized to produce phthalic anhydride, dyes, tanning agents, detergents. Further, methylnaphthalenes are used in the production of surfactants, agricultural products (e.g., including agricultural fluids), high-performing polymers, and aromatic solvents. Still further, the methyl groups in methylnaphthalenes can be useful for further functionalization. For example, 2,6 dimethylnaphthalene may undergo oxidation to produce dimethyl-2,6-naphthalene dicarboxylate (NDC) monomer that is utilized in the production of polyethylene naphthalate (PEN) polymer. As another example, a vinyl group can also be added to the methylnaphthalene and so that the resulting compound may be used as a monomer in various polymer applications.

Conventional processes for producing naphthalenes and methylnaphthalenes are often uneconomical. For example, both naphthalenes and methylnaphthalenes may be produced from coal-tar. However, the coal-tar distillation fraction that contains naphthalenes and methylnaphthalenes typically includes several co-boiling (or co-crystalizing) impurities, such as, for example, impurities containing indanes, indenes, phenols, nitrogen, and sulfur. Some of these impurities (e.g., nitrogen and sulfur containing impurities) can make up large percentages of the coal-tar distillation fraction. The subsequent removal of these co-boiling impurities often requires extensive hydro-treating and/or crystallization, which will often incur a considerable amount of energy and capital expenses. As another example, naphthalenes and methylnaphthalenes may be produced from other petroleum processes (e.g., reformer residue, heavy aromatic fractions); however, such streams suffer from limited availability and also typically include many of the same impurities mentioned above.

Accordingly, it would be desirable to produce naphthalenes and methylnaphthalenes in a more economically viable process. Preferable, such a process would utilize readily available, lower value and low sulfur streams.

### BRIEF SUMMARY

Some embodiments disclosed herein are directed to a process including providing an alkane-containing feed stream to a reactor. In addition, the process includes contacting the alkane-containing stream with an aromatization catalyst within the reactor. The aromatization catalyst comprises molecular sieve, and a dehydrogenation component. Further, the process includes producing a reactor effluent stream from the reactor. Still further, the process includes separating a product stream from the reactor effluent stream. The product stream comprises at least one or both of naphthalene and methylnaphthalene.

Other embodiments disclosed herein are directed to a process including providing a feed stream to a reactor. The feed stream includes a majority fraction of ethane. In addition, the process includes contacting the feed stream with an aromatization catalyst within the reactor. The aromatization catalyst comprises molecular sieve, and a dehydrogenation component. Further, the process includes producing a reactor effluent stream from the reactor, and separating the reactor effluent into a first stream and a second stream in a first separation unit. The first stream comprises hydrogen and methane, and the second stream comprises C₆-C₈ aromatic hydrocarbons, naphthalene, methylnaphthalenes. Still further, the process includes separating the second stream into third stream and a fourth stream in a second separation unit. The third stream comprises a majority of the C₆-C₈ aromatic hydrocarbons from the second stream, and the fourth stream comprises a majority of the naphthalene and methylnaphthalenes from the second stream. Also, the process includes separating the fourth stream into a product stream and a residue stream in a third separation unit. The product stream comprises one of a majority of the naphthalene from the fourth stream or a majority of the methylnaphthalenes from the fourth stream, and the residue stream comprises the other of a majority of the naphthalene from the fourth stream or a majority of the methylnaphthalenes from the fourth stream.

### BRIEF DESCRIPTION OF THE FIGURES

FIG. 1 is a flow diagram of a process and system for producing naphthalenes and methylnaphthalenes in accordance with at least some embodiments disclosed herein.

FIG. 2 is flow diagram of another process and system for producing naphthalenes and methylnaphthalenes in accordance with at least some embodiments disclosed herein.

FIG. 3 is another flow diagram of another process and system for producing naphthalenes and methylnaphthalenes in accordance with at least some embodiments disclosed herein.

### DETAILED DESCRIPTION

The following discussion is directed to various embodiments. However, it should be appreciated that the embodiments disclosed herein have broad application, and that the discussion of any embodiment is meant only to be exemplary of that embodiment, and not intended to suggest that the scope of the disclosure, including the claims, is limited to that embodiment. In the drawings, certain features and components herein may be shown exaggerated in scale or in somewhat schematic form and some details of conventional elements may not be shown in interest of clarity and conciseness. All documents described herein are incorporated by reference, including any priority documents and/or testing procedures, to the extent they are not inconsistent with this text. When numerical lower limits and numerical upper limits are listed herein, ranges from any lower limit to any upper limit are contemplated.

The term “conversion” when used in connection with a specified reactant in a reaction means the amount (weight basis) of the reactant consumed in the reaction. For example, when the specified reactant is ethane (C₂ hydrocarbon), 100% conversion means 100% of the ethane is consumed in the reaction. Conversion may also be indicative of the activity of a catalyst where a higher activity catalyst has a high conversion, and a low activity catalyst has a lower activity. The term “selectivity” refers to the production (on a weight basis) of a specified compound in a catalytic reaction. As an example, the phrase “a light hydrocarbon conversion reaction has a 100% selectivity for aromatic hydrocarbon” means that 100% of the light hydrocarbon (weight basis) that is converted in the reaction is converted to aromatic hydrocarbon. The term “yield” refers to the production of a specified compound or a class of compounds in a catalytic reaction, and is substantially equal to conversion times selectivity. The term “weight hourly space velocity”, referred to as “WHSV” means the quotient of the mass flow rate of the reactants divided by the mass of the catalyst. The term “constraint index” is defined in U.S. Pat. Nos. 3,972,832 and 4,106,218, both of which are incorporated herein by reference. As used herein, when a component (e.g., compound, element, etc.) of a stream or catalyst is said to comprise a “majority fraction” of that stream or catalyst, respectively, the component comprises greater than 50 wt. % of the stream or catalyst based on the total weight of the stream or catalyst, respectively. Reference to the periodic table or periodic table of elements are made with reference to the IUPAC (International Union of Pure and Applied Chemistry) Periodic Table. As used herein, the terms “about,” “approximately,” “substantially,” “around,” and the like mean+/−10%.

Embodiments disclosed herein include processes and systems for producing naphthalenes and/or methylnaphthalenes from an alkane-containing feed stream. In at least some embodiments, the alkane-containing feed stream comprises ethane. In at least some embodiments disclosed herein, at least some of the naphthalenes and methylnaphthalenes are produced from an aromatization reaction. The aromatization reaction(s) contemplated herein are performed by contacting the feed with one or more catalysts under specified conditions in order to produce the desired effluent stream. In order to facilitate further understanding on this process, the following description will focus on the various options for the catalyst of the aromatization reaction for at least some of the embodiments disclosed herein.

Specifically, in at least some embodiments the catalyst utilized in the aromatization reactions disclosed herein include a molecular sieve component, a dehydrogenation component, and a binder. The term “binder” as used in this description and appended claims includes matrix and other materials which are typically added during manufacturing of conventional molecular-sieve containing catalysts, e.g., (i) as a process-ability aid during extrusion, pelletizing, etc., and/or (ii) to make the catalyst more resistant to temperature and other process conditions employed during hydrocarbon conversion. The aromatization catalyst disclosed herein may contain ≤20 wt. % of binder in at least some embodiments, which may result in decreased selectivity to coke during aromatization, and an increase in run length before catalyst regeneration is needed.

In some embodiments, the aromatization catalyst is substantially free of binder, such as an inorganic binder. For example, the aromatization catalyst can contain ≤1 wt. % of binder, such as ≤0.1 wt. %, based on the weight of the catalyst. The molecular sieve component is typically present in the aromatization catalyst in an amount of >80 wt. %, or ≥90 wt. %, or ≥95 wt. %, or ≥98 wt. %, or ≥99 wt. %, based on the weight of the aromatization catalyst. In certain aspects, the molecular sieve component includes aluminosilicate, e.g., ≥90 wt. % of at least one aluminosilicate. The aluminosilicate can be an un-substituted aluminosilicate, a substituted aluminosilicate, or a combination thereof. For example, the aluminosilicate can be in a form where at least a portion of its original metal has been replaced, e.g., by ion exchange, with other suitable metal (typically metal cation) of Groups 1-13 of the Periodic Table. Typically, the aluminosilicate includes zeolite aluminosilicate, e.g., ≥90 wt. % of at least one zeolite based on the weight of the aluminosilicate. The term zeolite includes those in which at least part of the aluminum is replaced by a different trivalent metal, such as gallium or indium.

The molecular sieve component typically includes ≥90 wt. % of one or more of the specified molecular sieves, e.g., ≥95 wt. %, such as ≥99 wt. %. In certain aspects, the molecular sieve component includes at least one zeolite molecular sieve, e.g., ≥90 wt. % zeolite, such as ≥95 wt. %, based on the weight of the molecular sieve component. Although, the molecular sieve component can consist essentially of or even consist of zeolite, in alternative aspects the zeolite(s) is present in the molecular sieve component in combination with other (e.g., non-zeolitic) molecular sieve(s). The molecular sieve may be one that is in hydrogen form, e.g., one that has been synthesized in the alkali metal form, but is then converted from the alkali to the hydrogen form and has hydrogen ions. For example, zeolite can be present in hydrogen form, e.g., zeolite synthesized in the alkali metal form, but is then converted from the alkali to the hydrogen form.

In certain aspects, the molecular sieve component is a crystalline aluminosilicate and is present in the catalyst in an amount ≥80 wt. %, or ≥85 wt. %, or ≥90 wt. %, or ≥95 wt. % or ≥99 wt. %, based on the weight of the catalyst. When the molecular sieve component comprises crystalline aluminosilicate, when the crystalline aluminosilicate typically has a constraint index of less than 12, preferably, in the range of about 1 to about 12. Typically, the crystalline aluminosilicate is one having a medium pore size and a Constraint Index of less than or equal to about 12. Examples of suitable aluminosilicates include ZSM-5, ZSM-11, ZSM-12, ZSM-21, ZSM-22, ZSM-23, ZSM-35, ZSM-38, ZSM-48 ZSM-50, ZSM-57, and MCM-68, including mixtures and intermediates thereof such as ZSM-5/ZSM-11 admixture. ZSM-5 is described in U.S. Pat. No. 3,702,886 and Re. 29,948. ZSM-11 is described in U.S. Pat. No. 3,709,979. A ZSM-5/ZSM-11 intermediate structure is described in U.S. Pat. No. 4,229,424. ZSM-12 is described in U.S. Pat. No. 3,832,449. ZSM-21 is described U.S. Pat. No. 4,082,805. ZSM-22 is described in U.S. Pat. No. 4,556,477. ZSM-23 is described in U.S. Pat. No. 4,076,842. ZSM-35 is described in U.S. Pat. No. 4,016,245. ZSM-38 is described in U.S. Pat. No. 4,046,859. ZSM-48 is described in U.S. Pat. No. 4,234,231. ZSM-50 is described in U.S. Pat. No. 4,640,826. ZSM-57 is described in U.S. Pat. No. 4,873,067. TEA-Mordenite is described in U.S. Pat. Nos. 3,766,093 and 3,894,104. MCM-68 is described in U.S. Pat. No. 6,049,018.

The aluminosilicate's silica-to-alumina (Si:Al₂) atomic ratio is typically ≥2 molar, e.g., in the range of 10 to 300 molar, or in the range of from 5 to 100 molar. The silica-to-alumina ratio, Si:Al₂, is meant to represent the Si:Al₂ atomic ratio in the rigid anionic framework of the crystalline aluminosilicate. In other words, aluminum in (i) any matrix or binder, or (ii) in cationic, or other form within the crystalline aluminosilicate's channels is excluded from the Si:Al₂ atomic ratio. Aluminosilicates having a higher silica-to-alumina ratio can be utilized when a lower catalyst acidity is desired, e.g., in the range of from 44 to 100 molar, such as from 50 to 80 molar, or 55 to 75 molar.

In certain aspects, the crystalline aluminosilicate has a constraint index in the range of about 1 to 12 and is selected from the group consisting of a MCM-22 family material, ZSM-5, ZSM-11, ZSM-12, ZSM-21, ZSM-22, ZSM-23, ZSM-35, ZSM-38, ZSM-48, ZSM-50, ZSM-57, MCM-68 and mixtures of two or more thereof. Preferably, the aluminosilicate is ZSM-11 or H-ZSM-11 (the acidic form of ZSM-11), and more preferably, the aluminosilicate is ZSM-5 or H-ZSM-5 (the acidic form of ZSM-5). The molecular sieve can have a relatively small crystal size, e.g., small crystal ZSM-5, meaning ZSM-5 has a crystal size ≤0.05 μm, such as in the range of 0.02 μm to 0.05 μm. Small crystal ZSM-5 and the method for determining molecular sieve crystal sizes are disclosed in U.S. Pat. No. 6,670,517, which is incorporated by reference herein in its entirety.

For example, in other embodiments the crystalline aluminosilicate comprises at least one molecular sieve of the MCM-22 family, e.g., MCM-22 alone or in combination with other aluminosilicates, specified above, or other MCM-22 family materials. Materials of the MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), and ITQ-2 (described in International Patent Publication No. WO 97/17290), MCM-36 (described in U.S. Pat. No. 5,250,277), MCM-49 (described in U.S. Pat. No. 5,236,575), MCM-56 (described in U.S. Pat. No. 5,362,697) and mixtures of two or more thereof. Related aluminosilicates to be included in the MCM-22 family are UZM-8 (described in U.S. Pat. No. 6,756,030) and UZM-8HS (described in U.S. Pat. No. 7,713,513), both of which are also suitable for use as the molecular sieve component.

Besides the molecular sieve component, the aromatization catalyst further comprises a dehydrogenation component, e.g., an active metal. Even though both the dehydrogenation component and the molecular sieve can contain metal, the dehydrogenation component and the molecular sieve components are substantially different components, and each is present in the aromatization catalyst in the specified amounts. It should also be noted that even though it is believed that the specified active metals are effective for dehydrogenation, this might not always be the case, and the term “aromatization catalyst” should not be interpreted as requiring effectiveness for dehydrogenation when present in a particular aromatization catalyst. The dehydrogenation component typically comprises at least one element from Group 5 to 15 of the Periodic Table, and generally comprises one or more metallic elements in these groups. Typically ≥50 wt. % of the dehydrogenation component comprises the active metal, e.g., ≥75 wt. %, such as ≥90 wt. %, or ≥95 wt. %, based on the weight of the dehydrogenation component. Preferably, the active metal is a first active metal selected from the group consisting of and includes one or more of zinc (Zn), gallium (Ga), indium (In), copper (Cu), silver (Ag), tin (Sn), iron (Fe), cobalt (Co), nickel (Ni), gold (Au), manganese (Mn), chromium (Cr), molybdenum (Mo), tungsten (W), and mixtures of two or more thereof. Preferably, the first active metal is one or more of Mo, W, Zn, and Ga.

In some embodiments, the dehydrogenation component further comprises at least one second active metal in addition to the first active metal. The second active metal is different from the first active metal. The second active metal is selected from the group consisting of and includes one or more of phosphorus (P), platinum (Pt), palladium (Pd), lanthanum (La), rhenium (Re), and mixtures of two or more thereof. For example, the second active metal can be phosphorous.

In some embodiments, the aromatization catalyst contains at least about 0.005 wt. % of the first active metal, or in the range from about 0.005 wt. % to about 4.0 wt. %, or from about 0.01 wt. % to about 3.0 wt. %, based on the weight of the catalyst. When the second active metal is present, the aromatization catalyst can contain the second active metal in an amount, e.g., in a range from 0 wt. % to about 5.0 wt. %, or from about 0.005 wt. % to about 4.0 wt. %, or from about 0.01 wt. % to about 3.0 wt. %, based on the weight of the aromatization catalyst.

Without wishing to be bound by any particular theory, it is believed that the higher catalyst activity provided by the increased molecular sieve (preferably, aluminosilicate) content is modulated by the presence of the dehydrogenation component (e.g., the first active metal and optionally, the second active metal), leading to decreased coke selectivity and/or increased cycle length when the catalyst is used in a process for conversion of a light paraffinic hydrocarbon feedstock in a fixed bed reactor.

Although it is not required, the aromatization catalyst optionally includes binder (e.g., matrix) in an amount typically ≤20 wt. % based on the weight of the catalyst, e.g., ≤15 wt. %, such as ≤10 wt. %, or ≤5 wt. %, or ≤1 wt. %, or ≤0.1 wt. %. However, it should be appreciated that aromatization catalysts having greater than 20 wt. % of a binder are also contemplated. When used, binder can include inorganic material, e.g., clays and/or inorganic oxides. Suitable inorganic binders include alumina, silica, silica-alumina, titania, zirconia, magnesia, tungsten oxide, ceria, niobia, and mixtures of two or more thereof. The matrix can include naturally occurring materials and/or materials in the form of gelatinous precipitates or gels including mixtures of silica and metal oxides. Clays may also be included with the oxide type binders to modify the mechanical properties of the catalyst or to assist in its manufacture. Optionally, the binder may include one or more substantially inactive materials, e.g., diluent to control the amount of conversion so that products may be obtained economically and orderly without employing other means for controlling the rate of reaction. These materials may be incorporated into naturally occurring clays, e.g., bentonite and kaolin, to improve thermal and strength properties (e.g., crush strength) of the catalyst under catalytic conversion conditions. The binder may include other active materials, such as synthetic or naturally occurring aluminosilicate.

In some embodiments, the crystalline aluminosilicate and the active metal together comprise ≥85 wt. % of the aromatization catalyst, or ≥90 wt. %, or ≥95 wt. % or ≥99 wt. %. For example, the aromatization catalyst can be one where (i) ≥95 wt. % of the molecular sieve component comprises aluminosilicate, e.g., ≥99 wt. %, such as ≥99.9 wt. %, or even about 100 wt. %, (ii) ≥95 wt. % of the dehydrogenation component comprises active metal, e.g., ≥99 wt. %, such as ≥99.9 wt. %, or even about 100 wt. %, wherein the active metal is one or more of Ga, In, Zn, Cu, Re, Mo, and W, and (iii) the molecular sieve component and dehydrogenation component together constitute ≥95 wt. % of the aromatization catalyst, e.g., ≥99 wt. %, such as ≥99.9 wt. %, or even about 100 wt. %.

The aromatization catalyst can have any convenient form or morphology. For example, in certain aspects, the catalyst has the form of as-synthesized particles of the aromatization catalyst, e.g., a plurality of crystalline particles which comprise the crystalline molecular sieve and the active metal. The catalyst particles can be used as-synthesized. Alternatively or in addition, the catalyst particles can be, e.g., ground and sieved into a desired particle-size range; composited (with or without grinding and/or sieving) with binder to produce a solid catalytic body (e.g., a microstructure); self-bound to produce a microstructure; etc. The specified catalyst, as-synthesized, can be further processed (e.g., by grinding and sieving) into a wide range of sizes, e.g., (i) a powder having an average size ≤10 micrometers (“μm”), e.g., ≤2 μm, or ≤500 nm, or ≤200 nm, or ≤100 nm, or in the range of from 50 nm to 10 μm; (ii) a particulate having an average size >10 μm, e.g., >100 μm, such as >300 μm, or in the range of from 50 μm to 500 μm. Those having ordinary skill will appreciate that there is considerable overlap in terminology (powder vs. particulate) for catalysts having average sizes spanning the range from about 1 nm to about 1 mm. In other aspects, the specified catalyst has the form of a solid body or plurality thereof. For example, the specified catalyst can have the form of a macrostructure or plurality of macrostructures, including those having a size ≥0.01 mm in at least one dimension, e.g., ≥0.1 mm, or ≥1 mm. Examples of such macrostructures include those in the form of spheroids, extrudates (e.g., cylindrically-symmetric extrudates), pellets, fibers, thin films, etc. Conventional methods can be used for manufacturing a macrostructure comprising the specified catalyst, but the embodiments disclosed herein are not limited thereto. In certain aspects, the specified catalyst is self-bound, with the self-bound catalyst being in any convenient form during use for hydrocarbon conversion, e.g., in the form of a particulate which includes a plurality of self-bound catalyst particles, a self-bound porous macrostructure, a plurality of porous macrostructures, etc. Conventional methods can be used for making the specified catalyst in a self-bound form e.g., those disclosed in U.S. Pat. Nos. 5,510,016 and 6,787,623, which are incorporated by reference herein in their entireties, but the embodiments disclosed herein are not limited thereto. In any of the foregoing forms, e.g., crystalline particulate, bound composite, particulate of self-bound catalyst, self-bound macrostructure, etc., the specified catalyst typically has a surface area, as measured by nitrogen physisorption, in the range of from 100 m²/g to 600 m²/g, e.g., in the range of from 200 m²/g to 500 m²/g.

In at least some embodiments, ≥80 wt. % of the aromatization catalyst comprises (i) at least one active metal, and (ii) at least one molecular sieve having a framework of interconnected atoms. The framework defines an outer surface of the molecular sieve and a plurality of pores located within the molecular sieve. A feature of molecular sieve of these aspects is that at least some of the molecular sieve's pores have an average pore size in the range of from 4 Å (Angstroms) to 7 Å. Typically, the pores are not completely enclosed by the molecular sieve's outer surface. Instead, at least some of the pores typically have one or more pore openings through the outer surface. Another feature of the molecular sieve in these embodiments is that ≥90 wt. % of the active metal is located in those pores having an average pore size in the range of from 4 Å to 7 Å, e.g., ≥95 wt. %, such as ≥99 wt. %, or ≥99.9 wt. %. Yet another feature of the molecular sieve of these aspects is that ≤10 wt. % of the active metal is located proximate to the outer surface, e.g., ≤5 wt. %, such as ≤1 wt. %, or ≤0.1 wt. %. In this context, “proximate to” means within about 10 Å of the outer surface, e.g., within 5 Å, such as within 2.5 Å. More typically, the molecular sieve of these aspects has at least one set of pores of substantially uniform size extending through the molecular sieve, wherein geometric mean of the cross-sectional dimensions of each of the pores is >4 Å, or >5 Å, or >5.3 Å, e.g., ≥5.4 Å such as ≥5.5 Å, or in the range of 5 Å to 7 Å, or 5.4 Å to 7 Å.

Typically, the aromatization catalyst of at least some embodiments includes ≥90 wt. % of one or more molecular sieves and ≥0.005 wt. % of one or more of the active metals, e.g., ≥0.005 wt. % of one or more of the active metals in carbidic form, wherein the molecular sieve has a Constraint Index in the range of from 1-12, e.g., 2-11. When the molecular sieve and active metal together constitute less than 100 wt. % of the aromatization catalyst, ≥90 wt. % of the remainder of the aromatization catalyst can include binder, such as ≥99 wt. % of the remainder, provided the total amount of binder in the aromatization catalyst is ≤20 wt. % in some embodiments. A feature of the aromatization catalyst of these embodiments is that it includes ≤10 wt. % of metal in any form other than (A) framework metal if any (e.g., framework aluminum atoms) and (B) the active metal located in the pores of the molecular sieve. Since the active metal is primarily (e.g., more than 50 wt. %, such as more than 75 wt. %, or more than 90 wt. %) located in the pores of the molecular sieve, the aromatization catalyst of these embodiments can be referred to as an “embedded catalyst”. When the molecular sieve is a silicoaluminate such as ZSM-5, e.g., ZSM-5 in hydrogen form (“HZSM-5”), the embedded catalyst can be represented symbolically by [metal]@ZSM-5, [metal]@HZSM-5, and the like. For example, when the active metal is molybdenum and the molecular sieve is HZSM-5, the embedded catalyst can be represented symbolically by Mo@HZSM-5. This symbolic representation is different from that used to represent conventional metal-impregnated ZSM-5, e.g., Mo-impregnated ZSM-5, which typically has the symbolic representation Mo/ZSSM-5 or Mo-ZSM-5.

The conversion catalyst of at least some embodiments typically includes the molecular sieve in an amount ≥85 wt. %, based on the weight of the conversion catalyst, e.g., ≥90 wt. %, such as ≥95 wt. %, or in the range of from 85 wt. % to 99.9 wt. %. The molecular sieve of these aspects can include, e.g., ≥90 wt. % of at least one of the specified aluminosilicates.

Besides the molecular sieve, the catalysts of these embodiments includes at least one active metal in amount ≥0.005 wt. %, based on the weight of the catalyst. The active metal in these aspects is typically one or more of Ga, In, Zn, Cu, Re, Mo, W, La, Fe, Ag, Pt, and Pd, in neutral form and/or one or more oxides, sulfides and/or carbides of these metals. For example, the active metal can be one or more of Mo, Ga, Zn. The active metal can be, e.g., one or more carbidic molybdenum compounds, such as one or more molybdenum carbides and/or one or more molybdenum oxycarbides.

Typically, the aromatization catalyst of at least some embodiments includes ≥0.01 wt. % of the active metal, e.g., ≥0.1 wt. %, such as ≥0.5 wt. %, or ≥1 wt. %, or ≥5 wt. %, or ≥10 wt. %. These weight percents represent the weight of the metal, not the weight of the active-metal compound containing the active metal. For example, the aromatization catalyst can comprise ≥1 wt. % of Mo in the form of MoO₃, such as ≥5 wt. %, or ≥10 wt. %. In certain embodiments, (i) ≥99 wt. % of the active metal is one or more of Ga, Zn, Mo, and In, e.g., ≥99 wt. % of Mo in the form of one or more carbidic molybdenum compounds, such as one or 99 wt. % of Mo in the form of more molybdenum carbides and/or one or more molybdenum oxycarbides, and (ii) ≥99 wt. % of the molecular sieve is ZSM-S-type zeolite. One typical aromatization catalyst of these embodiments comprises ZSM-5, e.g., HZSM-5, and at least one of (i) molybdenum carbide, and (ii) molybdenum oxycarbide (“Catalyst X”), wherein the ZSM-5 and the molybdenum compound together comprise ≥80 wt. % of the aromatization catalyst, typically ≥90 wt. %, or ≥95 wt. %, or ≥99 wt. %. Mo is substantially the sole active metal of the catalyst.

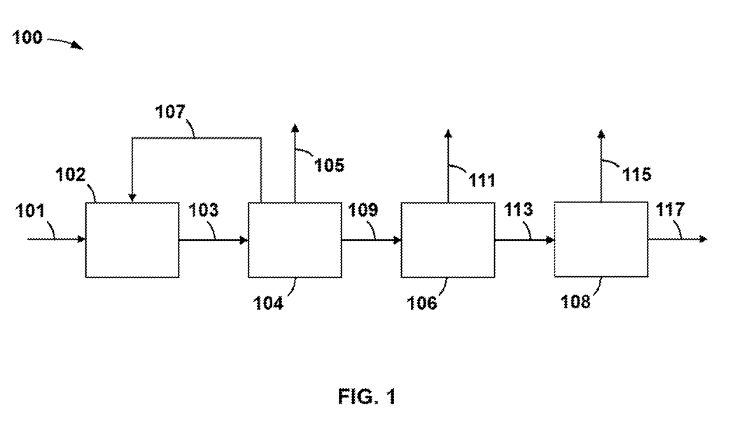

Referring now to FIG. 1, an embodiment of a system 100 and associated process for producing naphthalenes and/or methylnaphthalenes is shown. It should be appreciated that not all components necessary for carrying out the disclosed process may be shown in FIG. 1, and that in addition to the components and features shown, the embodiment of FIG. 1 may further include other components to facilitate the process disclosed herein (e.g., pumps, compressors, heat exchangers, tanks, etc.).

Initially, system 100 includes a feed stream 101 that is provided to an aromatization reactor 102. Feed stream 101 comprises C₂₊ alkanes (e.g., paraffins and/or olefins), and in at least some embodiments comprises ethane. For example, in some embodiments feed stream 101 comprises ethane in an amount ≥1 wt. %, e.g., ≥5 wt. %, such as ≥10 wt. % based on the total weight of feed stream 101. Suitable feed streams include those containing a majority fraction of ethane, e.g., >50 wt. % ethane, such as ≥75 wt. %, or ≥90 wt. %, or ≥95 wt. % based on the total weight of feed stream 101. For example, the feed stream 101 can comprise an amount of ethane in the range of from 1 wt. % to 100 wt. %, such as 5 wt. % to 95 wt. %, or 10 wt. % to 90 wt. %. One representative feed stream 101 comprises (i) ≥10 wt. % ethane, or ≥50 wt. %, or ≥90 wt. %, such as in the range of from 10 wt. % to 99.5 wt. % ethane, with ≥95 wt. % of the balance of the feedstock comprising one or more of methane, propane, and butanes. Thus, in at least some embodiments, feed stream 101 comprises a majority fraction of ethane. Feed stream 101 may be derived from any suitable source, such as, for example, a natural gas liquids pipeline, a naphtha hydro-treater, a cracking unit, a hydro-processing unit, etc.

As another example, in some embodiments, the feed stream 101 comprises one or more C₁ to C₉ non-aromatic hydrocarbon compounds, e.g., one or more light hydrocarbon (i.e., C₁ to C₅) compounds, such as one or more paraffinic light hydrocarbon compounds. For example, the feed stream 101 can comprise ≥1 wt. % based on the weight of the feed of one or more of (i) paraffinic C₂ to C₉ hydrocarbon, (ii) aliphatic C₁ to C₉ hydrocarbon, (iii) aliphatic paraffinic C₁ to C₉ hydrocarbon, (iv) paraffinic light hydrocarbon, (v) aliphatic light hydrocarbon, and (vi) aliphatic paraffinic light hydrocarbon; such as ≥10 wt. %, or ≥25 wt. %, or ≥50 wt. %, or ≥75 wt. %, or ≥90 wt. %, or ≥95 wt. %. Optionally, the feed further comprises diluent. Diluent present in the feed's source (e.g., methane and/or CO₂ present in natural gas) and diluent added to the feed are within the scope of the embodiments disclosed herein. Diluent, when present, is typically included in the feed in an amount ≤60 wt. % based on the weight of the feed, e.g., ≤50 wt. %, such as ≤40 wt. %, or ≤30 wt. %, or ≤20 wt. %, or ≤10 wt. %. A feed constituent is diluent when it is substantially non-reactive under the specified reaction conditions in the presence of the specified dehydrocyclization catalyst, e.g., methane, molecular nitrogen, and inert atomic gasses such as argon. Organic and inorganic diluents are within the scope of the embodiments disclosed herein.

The feed stream 101's non-aromatic C₁ to C₉ hydrocarbon can include aliphatic hydrocarbon, e.g., alkane. Representative alkane-containing feeds include those comprising at least 20 mole % of one or more C₁-C₉ alkane relative to the total number of moles in the feed, or at least 35 mole %, or at least 50 mole %, or at least 60 mole %, or at least 70 mole %, or at least 80 mole %. Additionally, or alternately, the alkane-containing feedstock can initially contain at least 50 mole % of one or more C₁-C₉ alkane relative to the total number of moles of hydrocarbon in the feed, or at least 60 mole %, or at least 70 mole %, or at least 80 mole %.

The feed stream 101 can include methane, e.g., ≥1 wt. % methane, such as ≥10 wt. %, or ≥20 wt. %, or ≥60 wt. %. When methane is substantially non-reactive under the specified aromatics formation reaction, the methane is considered diluent. Alternatively, or in addition to the methane and/or ethane, the feed can contain C₃ and/or C₄ hydrocarbons, e.g., (i) ≥20 wt. % propane, such as ≥40 wt. %, or ≥60 wt. %, and/or (ii) ≥20 wt. % butanes, such as ≥40 wt. %, or ≥60 wt. %. In some aspects, the feed can contain a reduced amount of C₅₊ hydrocarbon, e.g., ≤20 wt. %, such as ≤10 wt. % or ≤01 wt. %. In such aspects, the feed stream 101 can contain ≤10 wt. % of C₆₊ saturated hydrocarbon, e.g., ≤5 wt. %.

Optionally, the feed stream 101 comprises molecular hydrogen, e.g., ≥1 wt. % molecular hydrogen based on the weight of the feed, such as ≥5 wt. %. Optionally, the feed stream 101 contains unsaturated C₂₊ hydrocarbon, such as C₂-C₅ unsaturated hydrocarbon. When present, the amount of C₂₊ unsaturated hydrocarbon (e.g., C₂-C₅ unsaturated hydrocarbon) is typically ≤20 wt. %, e.g., ≤10 wt. %, such as ≤1 wt. %, or ≤0.1 wt. %, or in the range of from 0.1 wt. % to 10 wt. %. Typically, the feed stream 101 is substantially-free of aromatic hydrocarbon, where substantially-free in this context means an aromatic hydrocarbon concentration that is <1 wt. % based on the weight of the feed, such as ≤0.1 wt. %, or ≤0.01 wt. %, or ≤0.001 wt. %. Typically, the feed stream 101 comprises a total of ≤10 wt. % of impurities such as CO, CO₂, H₂S, and total mercaptan, e.g., ≤1 wt. % or ≤0.1 wt. %. One representative feed comprises 1 wt. % to 40 wt. % methane; ≥10 wt. % ethane, such as in the range of from 10 wt. % to 40 wt. %; 20 wt. % to 50 wt. % propane; and 20 wt. % to 50 wt. % butanes.

Light hydrocarbon in feed stream 101 can be obtained from one or more sources of hydrocarbon, e.g., from natural hydrocarbon sources such as those associated with producing petroleum, or from one or more synthetic hydrocarbons sources such as catalytic and non-catalytic reactions. Examples of such reactions include catalytic cracking, catalytic reforming, coking, steam cracking, etc. Synthetic hydrocarbon sources include those in which hydrocarbon within a geological formation has been purposefully subjected to one or more chemical transformations. The feed can include components recycled from the process, e.g., from one or more locations downstream of the aromatics formation.

In certain aspects, the source of light hydrocarbon includes natural gas, e.g., raw natural gas (“raw gas”). Natural gas is (i) a mixture comprising hydrocarbon, (ii) primarily in the vapor phase at a temperature of 15° C. and a pressure of 1.013 bar (absolute), and (iii) withdrawn from a geologic formation. Natural gas can be obtained, e.g., from one or more of petroleum deposits, coal deposits, and shale deposits. The natural gas can be one that is obtained by conventional production methods but the invention is not limited thereto. Raw natural gas is a natural gas obtained from a geologic formation without intervening processing, except for (i) treatments to remove impurities such as water and/or any other liquids, mercaptans, hydrogen sulfide, carbon dioxide; and (ii) vapor-liquid separation, e.g., for adjusting the relative amounts of hydrocarbon compounds (particularly the relative amounts of C₄₊ hydrocarbon compounds) in the natural gas; but not including (iii) fractionation with reflux. Conventional methods can be used for removing impurities and/or adjusting the relative amount of hydrocarbon compounds present in the feed, but the invention is not limited thereto. For example, certain components in the natural gas can be liquefied by exposing the natural gas to a temperature in the range of −57° C. to 15° C., e.g., −46° C. to 5° C., such as −35° C. to −5° C. At least a portion of the liquid phase can be separated in one or more vapor-liquid separators, e.g., one or more flash drums. One suitable raw natural gas comprises 3 mole % to 70 mole % methane, 10 mole % to 50 mole % ethane, 10 mole % to 40 mole % propane, 5 mole % to 40 mole % butanes and 1 mole % to 10 mole % of total C₅ to C₉ hydrocarbon. In certain aspects, ≥50 wt. % of the feed comprises natural gas, such as raw natural gas, e.g., ≥75 wt. %, or ≥90 wt. %, or ≥95 wt. %.

Any form of raw gas can be used as a source material, although the raw gas is typically one or more of (i) gas obtained from a natural gas well (“Gas Well”, Non-associated”, or “Dry” gas), (ii) natural gas obtained from a condensate well (“Condensate Well Gas”), and (iii) casing head gas (“Wet” or “Associated” gas). Table 1 includes typical raw gas compositional ranges (mole %) and, parenthetically, typical average composition (mole %) of certain raw gasses.

TABLE 1

| | Associated | | Dry | | Condensate | |
|---|---|---|---|---|---|---|
| Component | Gas | | Gas | | Well Gas | |
| CO₂ | 0-50 | (0.63) | 0-25 | (0) | 0-25 | (0) |
| N₂ | 0-50 | (3.73) | 0-25 | (1.25) | 0-25 | (0.53) |
| H₂S | 0-5 | (0.57) | 0-5 | (0) | 0-5 | (0) |
| CH₄ | 0-80 | (64.48) | 0-97 | (91.01) | 0-98 | (94.87) |
| C₂H₆ | 5-20 | (11.98) | 2-10 | (4.88) | 1-5 | (2.89) |
| C₃H₈ | 2-10 | (8.75) | 0.5-5 | (1.69) | 0.1-5 | (0.92) |
| i-butane | 0.1-5 | (0.93) | 0.05-1 | (0.14) | 0.1-5 | (0.31) |
| n-butane | 1-5 | (2.91) | 0.05-2 | (0.52) | 0.05-2 | (0.22) |
| i-pentane | 0.05-2 | (0.54) | 0.01-1 | (0.09) | 0.01-1 | (0.09) |

In certain aspects, the feed comprises ≥75 wt. % Associated Gas, based on the weight of the feed, e.g., ≥90 wt. % or ≥95 wt. %.

Within reactor 102, feed stream 101 is contacted with an aromatization catalyst under suitable conditions in order to convert at least some of the ethane and heavier alkanes (if present) within feed stream 101 into aromatic hydrocarbons (e.g., benzene, toluene, xylenes, etc.), naphthalenes, and methylnaphthalenes. The aromatization catalyst within reactor 102 is the same as any one or more of the aromatization catalysts previously described above. The feed stream 101 may be contacted with the aromatization catalyst within in at least one reaction zone within reactor 102. Each reaction zone may have one or more stages containing at least one bed of the aromatization catalyst. The catalyst bed may be one or more of a fixed bed, a moving bed, or fluidized bed. Conventional fixed, moving, and/or fluidized beds may be used in one or more of the reaction zones, but the embodiments disclosed here are not limited thereto. Although the aromatization catalyst can be present in the reaction zone in the form of a fluidized particulate, the process can alternatively (or in addition) be carried out with the aromatization catalyst in the form of a plurality of catalytic solid bodies (e.g., macrostructures such as extrudates, pellets, etc.).

Accordingly, in certain embodiments the aromatization catalyst resides in one or more fixed catalyst beds in one or more reaction zones within reactor 102 wherein feed stream 101 contacts the catalyst under the specified conversion conditions. In a fixed catalyst bed (also called a packed bed), the aromatization catalyst bed is “fixed” in the sense that it is substantially immobile with respect to the fixed reference frame during the conversion. The reactor 102 may be, for example, an adiabatic single bed, a multi-tube surrounded with heat exchange fluid or an adiabatic multi-bed with internal heat exchange, among others. At least one substantially similar second reaction zone may be operated in parallel with the first reaction zone, so that the first reaction zone may be operated in reaction mode while the second reaction zone is operated in regeneration mode to regenerate the second reaction zone's catalyst. Continuous operation may be carried out by alternating reaction and regeneration modes in the first and second reaction zones.

In other embodiments, the contacting within reactor 102 is carried out in a catalyst bed exhibiting catalyst motion during the process, e.g., in a moving bed, a fluidized bed, and ebullating bed, etc. In a moving catalyst bed, particles of the specified catalyst flow into and out of the reaction zone under the influence of an external force such as gravity. The catalyst particles substantially maintain their relative positions to one another during the flow, resulting in a movement of the bed with respect to the fixed reference frame. Average flow of the specified feedstock with respect to the catalyst flow may be concurrent, countercurrent, or cross-current.

In some embodiments, the reaction conditions within reactor 102 may include any suitable conditions, such as, for example, a temperature in the range of from about 400° C. to about 50° C., more preferably from about 400° C. to about 630° C., and still more preferably from about 500° C. to about 625° C. In addition, the process conditions within reactor 102 may include a pressure in the range of from about 35 kPaa (5 psia) to about 2200 kPaa (319 psia), more preferably from about 138 kPaa (20 psia) to about 2070 kPaa (300 psia), and still more preferably from about 207 kPaa (30 psia) to about 522 kPaa (80 psia). Further, the process conditions within reactor 102 may include a weight hourly space velocity (WHSV) greater than or equal to about 0.1 hr⁻¹, such as a WHSV of about 0.1 hr⁻¹ to about 20 hr⁻¹, more preferably from about 0.1 hr⁻¹ to about 10 hr⁻¹.

Reactor 102 produces an effluent stream 103 that comprises aromatic hydrocarbons (e.g., benzene, toluene, xylenes, etc.), naphthalene, and methylnaphthalenes. For example, in some embodiments, effluent stream 103 comprises from 10 to 40 wt. % aromatic hydrocarbons, from 2 to 30 wt. % naphthalene, and from 2 to 30 wt. % methylnaphthalenes, where each of the weight percentages above are given with respect to the total weight of effluent stream 103. In other embodiments, the process conditions and/or catalyst may be altered (e.g., by increasing temperature and/or lowering WHSV) to facilitate an increased selectivity for naphthalene and/or methylnaphthalenes within aromatization reactor 102.

In particular, in some embodiments, the residence time within reactor 102 may be optimized to result in 10 to 30 wt. % naphthalene in effluent stream 103. Specifically, in some embodiments, as reaction time increases, naphthalene production also increases to a relative maximum, and then decreases. Thus, by analyzing the specifics of the reaction in question, one may optimize the residence time of the feed stream (e.g., feed stream 101) within reactor 102 to allow the produced naphthalene to reach a maximum value. This increased selectivity toward naphthalene may be further facilitated by using a more acidic zeolite and/or using a Ga rather than Zn based catalyst and/or using a bimetallic catalyst.

In still other embodiments, the coke deposited on the catalyst within reactor 102 may be optimized to result in 10 to 30 wt. % methylnaphthalenes in effluent stream 103. Specifically, in some embodiments, as the amount of coke deposited on the aromatization catalyst increases, methylnaphthalene production also increases to a relative maximum, and then decreases. Thus, by analyzing the specifics of the reaction in question, one may optimize the amount of coke deposited on the catalyst to allow the produced methylnaphthalene to reach a maximum value. This increased selectivity toward methylnaphthalenes may be further facilitated by increasing the Ga content of the catalyst. Further, in some embodiments effluent stream 103 may additionally include light hydrocarbons (e.g., C₂-C₆) and unreacted feed stream components (e.g., unreacted ethane).

In at least some embodiments, most of any sulfur or nitrogen that is within feed stream 101 may be substantially removed in one or more sulfur and/or nitrogen removal units upstream of reactor 102. For example, in some embodiments, the feed to reactor 102 (i.e., feed stream 101) may have less than 20 ppm (by weight) sulfur and/or less than 500 ppm (by weight) nitrogen. Any suitable process or unit may be utilized to remove sulfur or nitrogen from feed stream 101 in these embodiments, such as, for example sulfur removal via amine adsorption, or cryogenic distillation of nitrogen. In addition, in some embodiments sulfur and/or nitrogen may be further removed from effluent stream 109 (e.g., via amine adsorption).

Upon exiting aromatization reactor 102, effluent stream 103 is provided to a first separation unit 104, which may comprise one or more separation devices (e.g., distillation columns, gravity based separators, etc.). In this embodiment, first separation unit 104 separates effluent stream 103 into a light stream 105 comprising hydrogen (H₂) and methane (CH₄), and a heavy stream 109 comprising aromatic hydrocarbons, naphthalene, methylnaphthalene, and other components that are liquid at ambient temperature and pressure conditions (e.g., benzene, alkylaromatic hydrocarbons, etc.). Light stream 105 is removed from system 100 and may be sold, disposed of (e.g., via a flare), utilized as fuel gas, and/or routed to another process (e.g., a pressure-swing adsorption may be used to recover the hydrogen, contained hydrogen could be utilized for hydrotreating of fuels streams, and/or the methane and/or hydrogen could be fed to a methanol synthesis process). Heavy stream 109 is then sent to a second separation unit 106.

In addition, in this embodiment separation unit 104 also produces a mid-stream 107 comprising an alkane fraction, which may further include unreacted ethane and/or the other light hydrocarbons (e.g., C₂-C₆) produced from reactor 102. This mid-stream 107 is recycled back to aromatization reactor 102 where it is again contacted with the aromatization catalyst under the conditions previously described above along with feed stream 101.

Second separation unit 106, like first separation unit 104, may comprise one or more separation devices (e.g., distillation columns, gravity based separators, etc.). In this embodiment, second separation unit 106 separates stream 109 into a light stream 111 comprising lighter aromatic hydrocarbons, such as, for example, benzene, toluene, xylenes (i.e., C₆-C₈ aromatic hydrocarbons), and a heavy stream 113 comprising naphthalene and methylnaphthalenes.

Light stream 111 may be sent to another processing unit for potential upgrading (e.g., para-xylene production, separation, and/or isolation processes such as transalkylation, alkylation, crystallization, selective adsorption, etc.). For example, light stream 111 may be further processed in a manner similar to that described in PCT/US2016/064928 (Attorney Docket Number 2016EM007-WO), the contents of which are incorporated herein by reference.

Referring still to FIG. 1, heavy stream 113 is routed from second separation unit 106 to third separation unit 108, which again may comprise one or more separation devices (e.g., distillation columns, crystallization, adsorption, etc.). Third separation device is configured to separate stream 113 into a residue stream 115 and a product stream 117. Separation unit 108 may be specifically configured to separate the desired one of naphthalene, methylnaphthalenes, or even a specific methylnaphthalene isomer into product stream 117 (e.g., so that product stream 117 comprises a majority fraction of either naphthalene or methylnaphthalenes, 1-methylnaphthalene, 2-methylnaphthalene), and separate the other of the two into residue stream 115. In particular, in at least some embodiments, product stream 117 may comprise at least 2 wt. % naphthalene or at least 2 wt. % methylnaphthalene based on the total weight of product stream 117, and in still other embodiments, product stream 117 may comprise at least 50 wt. % naphthalene or at least 50 wt. % methylnaphthalene based on the total weight of product stream 117. For example, if it is desired to produce naphthalene in product stream 117, third separation unit 108 may be configured to separate a majority of the methylnaphthalenes in heavy stream 113 into residue stream 115 and a majority of the naphthalene present in heavy stream 113 into the product stream 117. As another example, if it is desired to produce methylnaphthalenes in product stream 117, third separation unit 108 may be configured to separate a majority of the naphthalene present in heavy stream 113 into residue stream 115 and a majority of the methylnaphthalenes present in heavy stream 113 into product stream 117. For example, it is expected that naphthalene can be fractionated to reasonably high purity from methylnaphthalenes via a single distillation tower. However, regardless of whether it is desired to produce naphthalene or methylnaphthalenes in product stream 117, it should be appreciated that at least some naphthalene and methylnaphthalenes may be present within product stream 117 in some embodiments. In addition, it should be appreciated that in embodiments where methylnaphthalenes are produced into product stream 117, there may be one or more additional separation units downstream of separation unit 108 that remove heavier components that were not previously removed in separation units 104 and 106.

In addition, it should be appreciated that the liquid streams within system 100 that are downstream of reactor 102 (e.g., streams 109, 113, 111, 115, 117) would have a relatively low amount of sulfur and/or nitrogen due at least in part to the sulfur and/or nitrogen removal units upstream of reactor 102 (previously described above). For example, in some embodiments stream 109, 111, 113, 115, 117 may comprise less than 100 ppm sulfur and/or less than 100 ppm nitrogen. In still other embodiments, stream 109, 111, 113, 115, 117 may comprise less than 5 ppm sulfur and/or less than 5 ppm nitrogen.

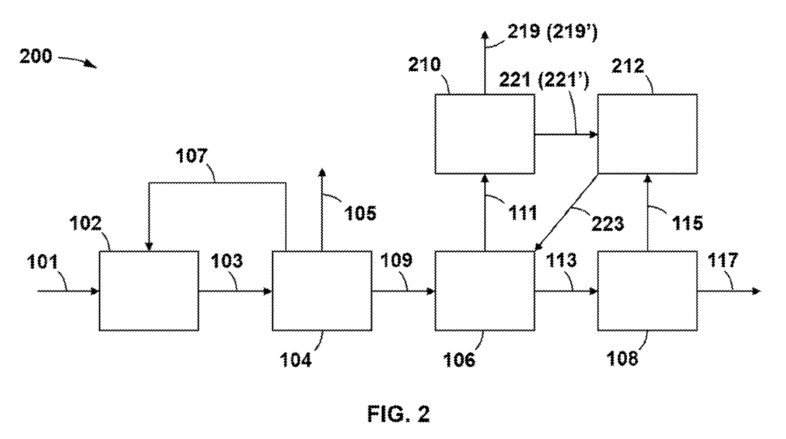

Referring now to FIG. 2, another embodiment of a system 200 and associated process for producing naphthalene and/or methylnaphthalenes is shown. System 200 is substantially the same as system 100, previously described, and thus, like components and features will be designated with like reference numerals and the description below will focus on the components and features of system 200 that are different from system 100.

In particular, as shown in FIG. 2, system 200 includes a transalkylation unit 212 that is configured to receive residue stream 115 and produce additional naphthalene or methylnaphthalenes that are further routed back to second separation unit 106 via stream 223. For example, in embodiments where it is desired to produce methylnaphthalene as a majority component or fraction in product stream 117, light stream 111 from second separation unit 106 is first routed to a fourth separation unit 210 that is configured to separate light stream 111 into a C₆₋ stream 219 and a C₇₊ stream 221. The C₆₋ stream 219 comprises, in at least some embodiments, a majority fraction of benzene, and the C₇₊ stream comprises a majority fraction of C₇-C₉ aromatic hydrocarbons (e.g., toluene, xylenes, ethylbenzene, etc.). The C₇-C₉ aromatic hydrocarbon-containing stream 221 is then routed to transalkylation unit 212 along with residue stream 115, which as previously described above, comprises naphthalene. Within transalkylation unit 212, the C₇-C₉ aromatic hydrocarbons and naphthalene react with one another in the presence of a transalkylation catalyst (discussed in more detail below) under suitable transalkylation conditions to allow akly groups from the C₇-C₉ aromatic hydrocarbons to transfer to the naphthalene molecules and therefore form methylnaphthalenes that are then flowed back to second separation unit 106 via stream 223.

The conditions within transalkylation unit 212 for producing methylnaphthalene from the C₇-C₉ aromatic hydrocarbon-containing stream 221 and the naphthalene-containing residue stream 115 may include a temperature in the range of 100 to 1000° C., preferably in the range of about 300 to about 500° C., a pressure in the range of about 790 to about 7000 kPa-a (kilo-Pascal absolute), preferably in the range of about 2170 to about 3000 kPa-a, a hydrogen to hydrocarbon (H₂:HC) molar ratio in the range of about 0 to about 20 (wherein hydrogen, if present, may be obtained from reactor 102 and separated from stream 105 via a separation unit—not shown), preferably in the range of 1-10, and a WHSV in the range of about 0.01 to about 100 hr⁻¹, preferably in the range of 1-20 hr⁻¹.

As another example, in embodiments where it is desired to produce naphthalene as a majority component or fraction in product stream 117, light stream 111 from second separation unit 106 is first routed to fourth separation unit 210 as previously described. However, in these embodiments separation unit 210 is configured to separate light stream 111 into a C₇₊ stream 219′ and a C₆₋ stream 221′. In this embodiment, the C₇₊ stream 219′ comprises, in at least some embodiments, C₇-C₉ aromatic hydrocarbons (e.g., toluene, xylenes, ethylbenzene, etc.), and the C₆₋ stream comprises a majority fraction of a majority fraction of benzene. The C₆₋ stream 221′ is then routed to transalkylation unit 212 along with residue stream 115. In this embodiment, residue stream 115 includes methylnaphthalenes that are reacted with the C₆-C₉ aromatic hydrocarbons (e.g., which may comprised mostly benzene as previously described) from light stream 111 in the presence of a transalkylation catalyst (discussed below) under suitable transalkylation conditions to allow akly groups from the methylnaphthalene molecules to transfer to the C₆-C₉ aromatic hydrocarbons and therefore form naphthalene that is then flowed back to second separation unit 106 via stream 223.

The conditions within transalkylation unit 212 for producing naphthalene from the C₆-C₉ aromatic hydrocarbon-containing stream 221 and the methylnaphthalene-containing residue stream 115 may be similar to the reaction conditions within transalkylation unit 212 for producing methylnaphthalene. For example, the reaction conditions within transalkylation unit 212 in these embodiments may include a temperature in the range of 100 to 1000° C., preferably in the range of about 250 to about 500° C., a pressure in the range of about 790 to about 7000 kPa-a (kilo-Pascal absolute), preferably in the range of about 2170 to about 3000 kPa-a, a hydrogen to hydrocarbon (H₂:HC) molar ratio in the range of about 0 to about 20, preferably in the range of 1-10, and a WHSV in the range of about 0.01 to about 100 hr⁻¹, preferably in the range of 1-20 hr⁻¹.

The transalkylation reactions discussed above may be conducted within any suitable reactor or reactors. For example, the above described transalkylation processes can be conducted in a radial flow reactor, fixed bed reactor, continuous down flow reactor, fluid bed reactor, or combination thereof.

In addition, the catalyst used in the transalkylation processes described above may be any suitable transalkylation catalyst or catalyst system, such as those disclosed in U.S. Publication Nos. 2016/0220987, 2016/0176786, 2016/0176787, the contents of which are incorporated herein by reference. For example, in some embodiments the transalkylation catalyst for use within transalkylation unit 212 may include a transalkylation component and/or at least one hydrogenation component, and optionally at least one inorganic binder.

In some embodiments, the transalkylation catalyst may comprise a catalyst system having multiple, preferably two or three, catalyst beds within transalkylation unit 212 (e.g., within at least one reactor within transalkylation unit 212). Each catalyst bed may accommodate a different catalyst. For example, in a two bed configuration, a first catalyst may reside in the upstream bed and second catalyst may reside in the downstream bed. Each catalyst may comprise a molecular sieve, either the same or different. At least one of the catalysts, and potentially each of the catalysts may comprise a hydrogenation component. Useful molecular sieves catalyst components include zeolite beta, zeolite Y, Ultrastable Y (USY), Dealuminized Y (Deal Y), Mordenite, NU-87, ZSM-3, ZSM-4 (Mazzite), ZSM-12, ZSM-18, MCM-22, MCM-36, MCM-49, MCM-56, EMM-10, EMM-10-P, ZSM-20, PSH-3, SSZ-25, ZSM-5, ZSM-11, ZSM-22, ZSM-23, ZSM-35, ZSM-48, ZSM-57 and ZSM-58.

Useful hydrogenation catalyst components may include W, vanadium (V), Mo, Re, Cr, Mn, Sn, a metal selected from Groups 6-10 of the Periodic Table of the Elements, or mixtures thereof. Additional examples of useful metals are Fe, ruthenium (Ru), osmium (Os), Ni, Co, rhodium (Rh), iridium (Ir), and noble metals such as Pt or Pd.

As an example, the first catalyst may comprise a molecular sieve from the group consisting of zeolite Y, zeolite X, USY, MCM-22, PSH-3, SSZ-25, ZSM-12, and zeolite beta, and a hydrogenation component comprising a metal selected from the group of Ga, Cu, Zn, Ni, Co, Rh, Pt, Pd, or Re, or combinations thereof, while the second catalyst comprises a molecular sieve of ZSM-5. In a specific example, the first catalyst comprises ZSM-12 and platinum while the second catalyst comprises ZSM-5. In an alternative example, the first catalyst comprises ZSM-12 and a hydrogenation component, the hydrogenation component comprising Pt and Sn while the second catalyst comprises ZSM-5.

As another example, the first catalyst may comprise a molecular sieve from the group consisting of at least one of ZSM-5, ZSM-11, ZSM-22, ZSM-23, ZSM-35, ZSM-48, ZSM-57 and ZSM-58 and a hydrogenation component comprising one metal selected from the group of Pt, Pd, or Re, or combinations thereof and the second catalyst comprises a molecular sieve from the group consisting of at least one of zeolite beta, zeolite Y, Ultrastable Y (USY), Dealuminized Y (Deal Y), Mordenite, NU-87, ZSM-3, ZSM-4 (Mazzite), ZSM-12, ZSM-18, MCM-22, MCM-36, MCM-49, MCM-56, EMM-10, EMM-10-P and ZSM-20, and a hydrogenation component comprising one metal selected from the group of Pt, Pd, or Re, or combinations thereof. In a further example in which three catalyst beds are utilized; the previous example may additionally include a third catalyst comprising a molecular sieve of ZSM-5. In yet a further example in which three catalyst beds are utilized; the previous three catalyst bed example's first and/or second catalyst may have a hydrogenation component comprising Pt and Sn.

Any or all of the transalkylation catalysts may also contain a binder or matrix material in an amount ranging from 5 to 95 wt. %, and typically from 10 to 60 wt. %. Preferably the binder or matrix material is resistant to the temperatures and other conditions employed in the transalkylation process. Such materials include active and inactive materials and synthetic or naturally occurring zeolites, as well as inorganic materials such as clays, silica and/or metal oxides such as alumina.

Additional details of the transalkylation catalyst system may be found in the patents and patent applications described in U.S. Pat. Nos. 5,942,651 and 7,663,010, and also U.S. Pat. Nos. 6,864,203; 6,893,624; 7,485,765; 7,439,204; 7,553,791; and 5,763,720. Each of these U.S. patents are incorporated herein by reference.

Thus, by including a transalkylation unit 212 downstream of the aromatization reactor 102, additional naphthalene or methylnaphthalenes may be produced in product stream 117. As a result, the overall efficiency of system 200 (i.e., in terms of the production yields of naphthalene or methylnaphthalenes from feed stream 101) may be enhanced over that produced from system 100 in FIG. 1.

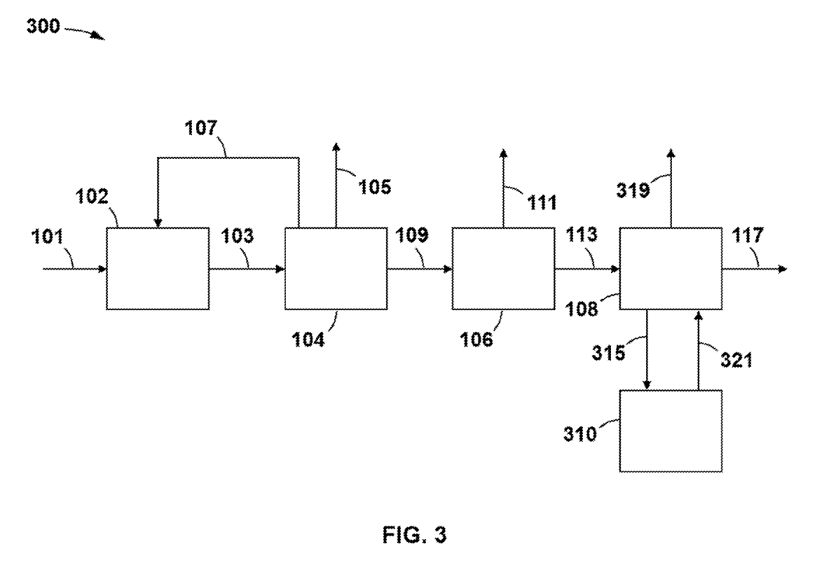

Referring now to FIG. 3, another embodiment of a system 300 and associated process for producing naphthalene is shown. System 300 is substantially the same as system 100, previously described, and thus, like components and features will be designated with like reference numerals and the description below will focus on the components and features of system 300 that are different from system 100.

In particular, in this embodiment, third separation unit 108 separates a residue stream 315 from heavy stream 113 that is rich in alkylnaphthalenes. For example, in some embodiments, residue stream 315 may comprise 20 to 100 wt. % alkylnaphthalenes, or more preferably 75 to 100 wt. % alkylnaphthalenes. In addition, the alkylnaphthalenes in residue stream 315 may include, for example, methylnaphthalenes, dimethylnaphthalenes, acenaphthalene, ethylnaphthalenes, and/or other C₁₀₊ hydrocarbons. Further, when residue stream 315 is produced from third separation unit 108 as in FIG. 3, a light stream 319 comprising hydrogen (H₂), light gases, and liquids is also produced from third separation unit 108. Light stream 319 may comprise a liquid component, but this liquids component includes a lesser or reduced amount of naphthalene and methylnaphthalenes. Specifically, in these embodiments, light stream 319 comprises less than 10 wt. % naphthalene and less than 10 wt. % methylnaphthalenes. More particularly, in some of these embodiments, light stream 319 comprises from about 0 to about 2 wt. % naphthalene and from about 0 to about 1 wt. % methylnaphthalenes.

Alkylnaphthalene-containing residue stream 315 is routed to a hydrodealkylation reactor 310 where it is contacted with a metal loaded acid catalyst (which may be referred to herein as a hydrodealkylation catalyst) in the presence of hydrogen to remove alkyl groups from the alkylnaphthalenes (e.g., such as methylnaphthalene) and form additional naphthalenes, which are then provided back to third separation unit 108 via a hydrodealkylated stream 321 (or hydrodealkylation stream 321). Hydrodealkylated stream 321 comprises a higher amount of naphthalene than the residue stream 315 and in at least some embodiments comprises a majority fraction of naphthalene. For example, hydrodealkylated stream 321 comprises at least 50 wt. % naphthalene, or from about 50 to about 97 wt. % naphthalene, based on the total weight of hydrodealkylated stream 321. By comparison, in at least some embodiments, residue stream 315 may comprise less than or equal to 20 wt. % naphthalene, or from about 0.1 to about 20 wt. % naphthalene, based on the total weight of residue stream 315. Thus, by utilizing a hydrodealkylation reactor 310, alkylnaphthalenes (e.g., methylnaphthalene) formed during the aromatization reaction within reactor 102 may be converted into naphthalene so that the overall yield of naphthalene may be increased within product stream 117 in these embodiments.

Any suitable catalyst may be used within hydrodealkylation reactor 310. For example, reactor 310 may include any one or more of the catalysts described in U.S. Pat. No. 4,982,040, the contents of which are incorporated herein by reference. For example, the catalyst utilized within the hydrodealkylation reactor 310 may comprise MCM-22, ZSM-5, PSH-3 (described in U.S. Pat. No. 4,439,409), etc. Furthermore the catalyst of hydroalkylation reactor 310 may include one or more metals (e.g., W, V, Mo, Re, Ni, Co, Cr, Mn, Pt, Pd, etc.).

In addition, the conditions within reactor 310 may include a temperature from about 300° C. to about 675° C., and preferably from about 375° C. to about 575° C., a pressure of from about atmospheric to about 2000 psig and preferably from about 20 to about 1000 psig and a WHSV of from about 0.1 to about 500 hr⁻¹ and preferably from about 0.5 to about 100 hr⁻¹.

Optionally, the hydrogen provided to hydrodealkylation reactor 310 may be taken from light stream 105 produced from first separation unit 104. Alternatively, some or all of the hydrogen provided to hydroalkylation reactor 310 may be obtained from an external source (e.g., from an external pipeline, process, tank, etc.). In addition, any excess hydrogen, light gases and liquids with lower naphthalene and methylnaphthalene content may be removed from system 300 via light stream 319. It should also be appreciated that any C2+ hydrocarbons in light stream 319 may be routed to reactor 102.

As a result of the embodiments disclosed herein, naphthalene and/or methylnaphthalenes may be produced from a relatively abundant feed stream (e.g., ethane-containing streams) through use of an aromatization reactor. In addition, as described above the yield of naphthalene and methylnaphthalenes may be increased by incorporating transalkylation and/or hydrodealkylation reactions downstream of the main aromatization reactor.

Particular reference will now be made to the following non-limiting examples.

### Example 1

Ethane was flowed through a reactor containing a Ga-ZSM-5 catalyst at a temperature of 630° C., a pressure of 15 psig, and WHSV 5 hr⁻¹. The Ga content of the catalyst was about 3 wt. % based on the total weight of the catalyst. The first product sample was analyzed in a gas chromatograph at 3 minutes time on stream. The ethane conversion rate was found to be 44.7%. In addition, the hydrocarbon selectivities of naphthalene, 1-methyl naphthalene, and 2-methyl naphthalene were found to be 15.0%, 5.2%, and 10%, respectively.

### Example 2

Ethane was flowed through a reactor containing the same catalyst and under the same conditions as described above in Example 1. The reaction was carried out for a total of 2-3 hours followed by regeneration. A number of these reaction-regeneration cycles were carried out to collect a liquid sample. The collected liquid sample was analyzed using gas chromatograph and mass spectroscopy. The hydrocarbon selectivities of naphthalene, 1-methyl naphthalene, and 2-methyl naphthalene were found to be 15.2%, 8.1%, and 15.1%, respectively, in the liquid sample.

### Example 3

An experiment was conducted to determine the effectiveness of a transalkylation reaction to form additional naphthalenes or methylnaphthalenes (e.g., such as discussed above for the embodiment of FIG. 2). A feed containing a mixture of 124-trimethylbenzene (as an alkylating agent) with naphthalene was fed to a down flow fixed bed reactor containing a formulated USY extrudate catalyst (80 wt. % zeolite/20 wt. % alumina binder—wherein each of the given weight percentages are given relative to the total weight of the catalyst). The feed included a 9:1 ratio of 124-trimethylbenezene to naphthalene, and the reaction conditions included a temperature of 350° C., a pressure of 600 psig, and WHSV of 4.0 hr⁻¹. The liquid product from the reactor was analyzed by a 6890 Agilent gas chromatograph. The naphthalene conversion rate was found to be 44%. In addition, after 5 days of testing, the selectivity to methylnaphthalenes was found to be 68% while the selectivity to dimethylnaphthalenes was found to be 18%.

It is expected that high naphthalene conversion and high methylnaphthalene and dimethylnaphthalene selectivity can be achieved with other zeolite catalyst (i.e., other than the USY extrudate discussed above), such as, for example beta and/or MCM-49. Also, it is also expected that high naphthalene conversion and high methylnaphthalene and dimethylnaphthalene selectivity can be achieved using feed containing, toluene, xylenes, and/or alkylbenzenes as an alkylating agent.

While various embodiments have been disclosed herein, modifications thereof can be made without departing from the scope or teachings herein. In particular, many variations and modifications of the systems, apparatus, and processes described herein are possible and are within the scope of the disclosed subject matter. Accordingly, embodiments disclosed herein are exemplary only and are not limiting. As a result, the scope of protection is not limited to the embodiments described herein, but is only limited by the claims that follow, the scope of which shall include all equivalents of the subject matter of the claims. Unless expressly stated otherwise, the steps in a method claim may be performed in any order. The use of identifiers such as (a), (b), (c) before steps in a method claim is not intended to and does not specify a particular order to the steps. Rather the use of such identifiers are used to simplify subsequent reference to such steps. Finally, the use of the term “including” in both the description and the claims is used in an open ended fashion, and should be interpreted as meaning “including, but not limited to.”

1. A process, comprising:
(a) providing an alkane-containing feed stream to a reactor;
(b) contacting the alkane-containing stream with an aromatization catalyst within the reactor, wherein the aromatization catalyst comprises molecular sieve, and a dehydrogenation component;
(c) producing a reactor effluent stream from the reactor; and
(d) separating a product stream from the reactor effluent stream, wherein the product stream comprises at least one or both of naphthalene and methylnaphthalene.

2. The process of claim 1, wherein the alkane-containing feed comprises a majority fraction of ethane.

3. The process of claim 2, wherein the molecular sieve of the aromatization catalyst comprises ZSM-5.

4. The process of claim 3, wherein the dehydrogenation component comprises gallium.

5. The process of claim 4, further comprising:
(e) removing a majority of one or both of sulfur or nitrogen in the feed stream before providing the feed stream to the reactor in (a).

6. The process of claim 5, wherein the reactor effluent stream contains one or both of:
less than 5 ppm sulfur; and
less than 5 ppm of nitrogen.

7. The process of claim 6, wherein the contacting in (b) is carried out with a temperature of about 500° C. to about 625° C., a pressure of about 207 kPaa (30 psia) to about 522 kPaa (80 psia), and a WHSV of about 0.1 hr⁻¹ to about 10 hr⁻¹.

8. The process of claim 7, wherein the product stream comprises at least 50 wt. % naphthalene, based on the total weight of the product stream.

9. The process of claim 7, wherein the product stream comprises at least 50 wt. % methylnaphthalenes, based on the total weight of the product stream.

10. The process of claim 9, further comprising:
(f) separating a residue stream from the reactor effluent stream, wherein the residue stream comprises at least one of naphthalene and methylnaphthalenes;
(g) transalkylating the residue stream to produce a transalkylation effluent stream, wherein the transalkylation effluent stream comprises more naphthalene or more methyl naphthalene than the residue stream; and
(h) recycling at least some of the transalkylation effluent stream to the separating in (d).

11. The process of claim 10, wherein the product stream comprises a majority fraction of naphthalene, wherein the residue stream comprises methylnaphthalenes, and wherein the process further comprises:
(i) separating a C₆ aromatic hydrocarbon-containing stream from the reactor effluent stream; and
(j) flowing the C₆ aromatic hydrocarbon-containing stream to the transalkylating in (g);
wherein (g) further comprises contacting the C₆ aromatic hydrocarbon-containing stream with the residue stream in the presence of a transalkylation catalyst to produce the transalkylation effluent stream, and
wherein the transalkylation effluent comprises more naphthalene than the residue stream.

12. The process of claim 10, wherein the product stream comprises a majority fraction of methylnaphthalenes, wherein the residue stream comprises naphthalene, and wherein the process further comprises:
(k) separating a C₇-C₉ aromatic hydrocarbon-containing stream from the reactor effluent stream; and
(l) flowing the C₇-C₉ aromatic hydrocarbon-containing stream to the transalkylating in (g);
wherein (g) further comprises contacting the C₇-C₉ aromatic hydrocarbon-containing stream with the residue stream in the presence of a transalkylation catalyst to produce the transalkylation effluent stream, and
wherein the transalkylation effluent comprises more methylnaphthalene than the residue stream.

13. The process of claim 12, wherein the transalkylation catalyst comprises an Ultrastable Y (USY) extrudate.

14. The process of claim 9, further comprising:
(m) separating an alkylnaphthalenes-containing stream from the reactor effluent stream;
(n) hydrodealkylating the alkylnaphthalenes in the alkylnaphthalenes-containing stream to produce a hydrodealkylation effluent stream that comprises a greater amount of naphthalene than the alkylnaphthalenes-containing stream; and
(o) recycling the hydrodealkylation effluent stream to the separating in (d).

15. The process of claim 14, wherein the product stream comprises a majority fraction of naphthalene.

16. The process of claim 14, wherein (n) further comprises contacting the alkylnaphthalenes-containing stream with a hydrodealkylation catalyst in the presence of hydrogen, wherein the hydrodealkylation catalyst comprises a molecular sieve selected from the group consisting of MCM-22, ZSM-5, and PSH-3.

17. The process of claim 16, further comprising:
(p) separating hydrogen from the reactor effluent stream; and
(q) providing the hydrogen to the hydroalkylating in (n).

18. A process, comprising:
(a) providing a feed stream to a reactor, wherein the feed stream comprises a majority fraction of ethane;
(b) contacting the feed stream with an aromatization catalyst within the reactor, wherein the aromatization catalyst comprises molecular sieve, and a dehydrogenation component;
(c) producing a reactor effluent stream from the reactor;
(d) separating the reactor effluent into a first stream and a second stream in a first separation unit, wherein the first stream comprises hydrogen and methane, and wherein the second stream comprises C₆-C₈ aromatic hydrocarbons, naphthalene, methylnaphthalenes;
(e) separating the second stream into third stream and a fourth stream in a second separation unit, wherein the third stream comprises a majority of the C₆-C₈ aromatic hydrocarbons from the second stream and wherein the fourth stream comprises a majority of the naphthalene and methylnaphthalenes from the second stream; and
(f) separating the fourth stream into a product stream and a residue stream in a third separation unit, wherein the product stream comprises one of a majority of the naphthalene from the fourth stream or a majority of the methylnaphthalenes from the fourth stream, and wherein the residue stream comprises the other of a majority of the naphthalene from the fourth stream or a majority of the methylnaphthalenes from the fourth stream.

19. The process of claim 18, further comprising:
(g) providing the residue stream and at least a portion of the second stream to a transalkylation unit;
(h) contacting the residue stream and the at least a portion of the second stream with a transalkylation catalyst within the transalkylation unit;
(i) producing a transalkylation effluent stream from the transalkylation unit that comprises either a greater amount of naphthalene or a greater amount of methylnaphthalenes than the residue stream; and
(j) recycling the transalkylation effluent stream to the second separation unit.

20. The process of claim 18, wherein the product stream comprises a majority of the naphthalene from the fourth stream, wherein the residue stream comprises a majority of the methylnaphthalenes from the fourth stream, and wherein the process further comprises:
(k) providing the residue stream to a hydrodealkylation unit;
(l) contacting the residue stream with a hydrodealkylation catalyst;
(m) producing a hydrodealkylation effluent stream that comprises a greater amount of naphthalene than the residue stream; and
(n) recycling the hydrodealkylation effluent stream to the third separation unit.
